(19) United States
(12) Patent Application Publication
(10) Pub. No.: US 2025/0152878 A1
(43) Pub. Date: May 15, 2025

(54) TURBINE BOX AND VENTILATOR

(71) Applicant: RESVENT MEDICAL TECHNOLOGY CO., LTD, Shenzhen (CN)

(72) Inventors: Wengui Zhang, Shenzhen (CN); Rongrong Zhu, Shenzhen (CN)

(73) Assignee: RESVENT MEDICAL TECHNOLOGY CO., LTD, Shenzhen (CN)

(21) Appl. No.: 18/820,536

(22) Filed: Aug. 30, 2024

(30) Foreign Application Priority Data
Nov. 14, 2023 (CN) 2023115945879

(51) Int. Cl. A61M 16/00; A61M 16/10

(52) U.S. Cl. CPC A61M 16/0066; A61M 16/105; A61M 2205/42

(57) ABSTRACT

A turbine box provided includes a main body containing a resonant silencing structure, which includes a resonance plate, a resonance chamber wall, and a silencing chamber formed by the resonance plate and the resonance chamber wall. A turbine blower is mounted in the silencing chamber installs, the silencing chamber is open to outside to receive external airflow, and under an action of the turbine blower, the external airflow is transformed into an airflow with a predetermined flow rate. The resonance plate defines a first hole and a second air hole located on opposite sides of the turbine blower; an airflow noise generated by the external airflow entering the silencing chamber, along with an vibration noise generated by the turbine blower, induce a resonance effect between the resonance plate and the resonance chamber wall to dampens both the airflow noise and the vibration noise.

This non-provisional patent application claims priority under 35 U.S.C. § 119 from Chinese Patent Application No. 202311594587.9 filed on Nov. 24, 2023, the entire content of which is incorporated herein by reference.

### TECHNICAL FIELD

This application relates to ventilator technologies, particularly to a turbine box, and a ventilator.

### BACKGROUND

Ventilators are precision medical devices with complex structures. A turbine is the core component of each ventilator. Inside the turbine, there is a fan driven by an electric motor, responsible for exhaust and ventilation. In the demanding environment of competitive ICUs, the turbine must maintain high precision, high reliability, and low noise levels. However, the high-speed rotation of the turbine fan during ventilator operation can generate significant vibration noises from wind and vibration, seriously affecting the recovery of patients in the ICU. The main technical solutions adopted by existing products are as follows:

There are two typical solutions to address ventilator noise. The first solution utilizes a fully wrapped sound-absorbing cotton method. In this solution, first, the turbine (the primary noise source) is completely wrapped with sound-absorbing cotton; second, the turbine is then positioned centrally within a housing; third, filling remaining internal spaces of the housing with sponge. The first solution effectively reduces noise by harnessing the sound-absorbing properties of the cotton and the sponge within the housing. The second solution employs an airway wrapping technique, which involves strategically positioning specially designed sound-absorbing materials at the rear ends of the turbine's inlet and outlet ducts. By utilizing the sound-absorbing capabilities of these materials, a noise reduction is achieved.

However, the existing solutions primarily rely on sound-absorbing cotton to reduce noise, which is effective primarily in absorbing turbine noise within the mid to high frequency bands. Nevertheless, they are essentially unable to absorb low and mid-frequency noise. Although a noise reduction effect is observed across the entire ventilator, the results are not entirely SUMMARY

In a first aspect, there is provided a turbine box, the turbine box include a main body, a resonance plate, and a resonance chamber wall. The main body defining a first silencing chamber, a second silencing chamber, a third silencing chamber, an air inlet, and an air outlet. The first silencing chamber is communicated with the air inlet; the third silencing is communicated with the air outlet, each of the first silencing chamber and the second silencing is configured to accommodate a noise reduction assembly, the third silencing chamber is configured to accommodate a turbine blower, external airflow enters to the main body, and passes sequentially through the first silencing chamber, the second silencing chamber, and the third silencing chamber before entering the turbine blower; under an action of the turbine blower, the external airflow is transformed into an airflow with a target flow rate; a resonance plate, defining a first air hole and a second air hole allowing the external airflow to flow from the second silencing chamber into the third silencing chamber; the first air hole and second air hole being located on opposite sides of the turbine blower. The resonance chamber wall surrounds the third silencing chamber, and together with the resonance plate define the third silencing chamber, an airflow noise is generated by the external airflow entering the third silencing chamber through the first air hole and the second air hole, as well as an vibration noise of the turbine blower, cause the resonance plate and the resonance chamber wall to form a resonance silencing structure to silence the airflow noise and the vibration noise.

In a second aspect, there is provided a ventilator, the ventilator includes a ventilator body, and a turbine box mounted to the ventilator body. the turbine box include a main body, a resonance plate, and a resonance chamber wall. The main body defining a first silencing chamber, a second silencing chamber, a third silencing chamber, an air inlet, and an air outlet. The first silencing chamber is communicated with the air inlet; the third silencing is communicated with the air outlet, each of the first silencing chamber and the second silencing is configured to accommodate a noise reduction assembly, the third silencing chamber is configured to accommodate a turbine blower, external airflow enters to the main body, and passes sequentially through the first silencing chamber, the second silencing chamber, and the third silencing chamber before entering the turbine blower; under an action of the turbine blower, the external airflow is transformed into an airflow with a target flow rate; a resonance plate, defining a first air hole and a second air hole allowing the external airflow to flow from the second silencing chamber into the third silencing chamber; the first air hole and second air hole being located on opposite sides of the turbine blower. The resonance chamber wall surrounds the third silencing chamber, and together with the resonance plate define the third silencing chamber, an airflow noise is generated by the external airflow entering the third silencing chamber through the first air hole and the second air hole, as well as an vibration noise of the turbine blower, cause the resonance plate and the resonance chamber wall to form a resonance silencing structure to silence the airflow noise and the vibration noise.

In a third aspect, there is provided a turbine box. The turbine box includes a resonance plate, a resonance chamber wall, and a silencing chamber formed by the resonance plate and the resonance chamber wall, the silencing chamber is configured to install a turbine blower, the silencing chamber being open to outside to receive external airflow, and under an action of the turbine blower, the external airflow is transformed into an airflow with a target flow rate, wherein the resonance plate defines a first hole and a second air hole; the first and second air holes are located on opposite sides of the turbine blower; an airflow noise generated by the external airflow entering the silencing chamber through the first air hole and the second air hole, along with an vibration noise generated by the turbine blower, induce a resonance effect between the resonance plate and the resonance chamber wall to dampens both the airflow noise and the vibration noise.

The above-mentioned turbine box has a resonance silencing structure, which suppresses airflow noise and turbine blower vibration noise, thereby filtering out mid to low frequency noise.

### BRIEF DESCRIPTION OF THE DRAWINGS

In order to provide a clearer explanation of the embodiments or technical solutions in the present application or prior art, a brief introduction will be given below to the accompanying drawings required in the embodiments or prior art description. It is evident that the accompanying drawings in the following description are only some embodiments of the present application. For those skilled in the art, other accompanying drawings can be obtained based on the structures shown in these drawings without creative labor.

FIG. 1 is a three-dimensional exploded view of a turbine assembly provided in an embodiment of the present application.

FIG. 2 is a three-dimensional schematic diagram of the turbine assembly shown in FIG. 1.

FIG. 3 is a three-dimensional exploded view of the turbine box of the turbine assembly shown in FIG. 1.

FIG. 4 is another exploded view of the turbine box shown in FIG. 3.

FIG. 5 is another three-dimensional schematic diagram of the turbine box shown in FIG. 3.

FIG. 6 is a three-dimensional exploded view of the turbine assembly at another angle as shown in FIG. 1.

FIG. 7 is a schematic sectional view of the turbine assembly shown in FIG. 1.

FIG. 8 is another angle sectional view of the turbine assembly shown in FIG. 1.

The implementation, functional characteristics, and advantages of the purpose of this application will be further explained in conjunction with the embodiments, with reference to the accompanying drawings.

### DETAILED DESCRIPTION OF THE EMBODIMENTS

In order to make the purpose, technical solution, and advantages of this application clearer and clearer, the following will provide further detailed explanations of this application in conjunction with the accompanying drawings and embodiments. It should be understood that the specific embodiments described here are only intended to explain the present application and are not intended to limit the present application. Based on the embodiments in this application, all other embodiments obtained by ordinary technical personnel in this field without creative labor fall within the scope of protection of this application.

The terms “first”, “second”, “third”, “fourth”, etc. (if any) in the specification and claims of this application, as well as the accompanying drawings, are used to distinguish similar planning objects and do not need to be used to describe specific order or sequence. It should be understood that the data used in this way can be interchanged in appropriate cases, in other words, the described embodiments are implemented in order other than those illustrated or described herein. In addition, the terms “including” and “having”, as well as any variations thereof, may also include other content, such as processes, methods, systems, products, or equipment that include a series of steps or units, not necessarily limited to those clearly listed, but may include other steps or units that are not clearly listed or inherent to these processes, methods, products, or equipment.

It should be noted that the descriptions related to “first”, “second”, etc. in this application are only for descriptive purposes and cannot be understood as indicating or implying their relative importance or implying the quantity of technical features indicated. Therefore, the features limited to “first” and “second” may explicitly or implicitly include one or more of the said features. In addition, the technical solutions between various embodiments can be combined with each other, but must be based on what ordinary technical personnel in the art can achieve. When the combination of technical solutions conflicts or cannot be achieved, it should be considered that the combination of such technical solutions does not exist and is not within the scope of protection required by this application.

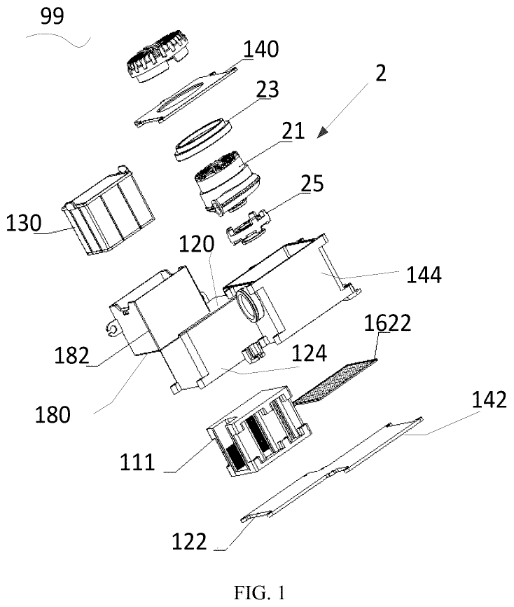

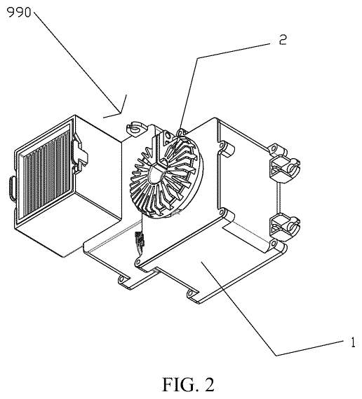

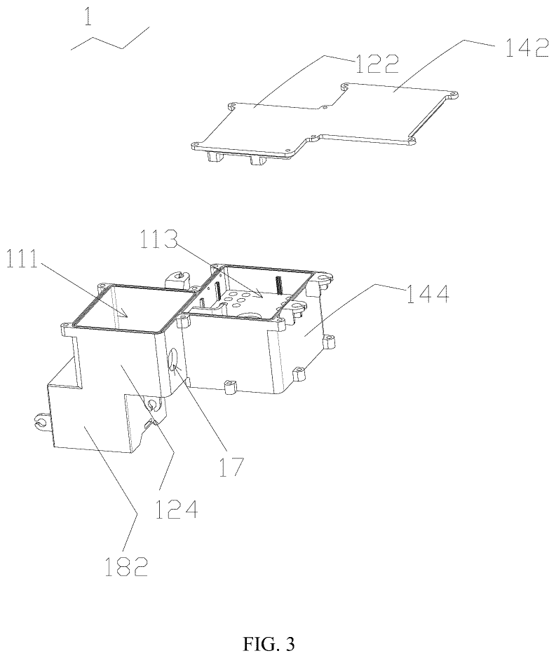

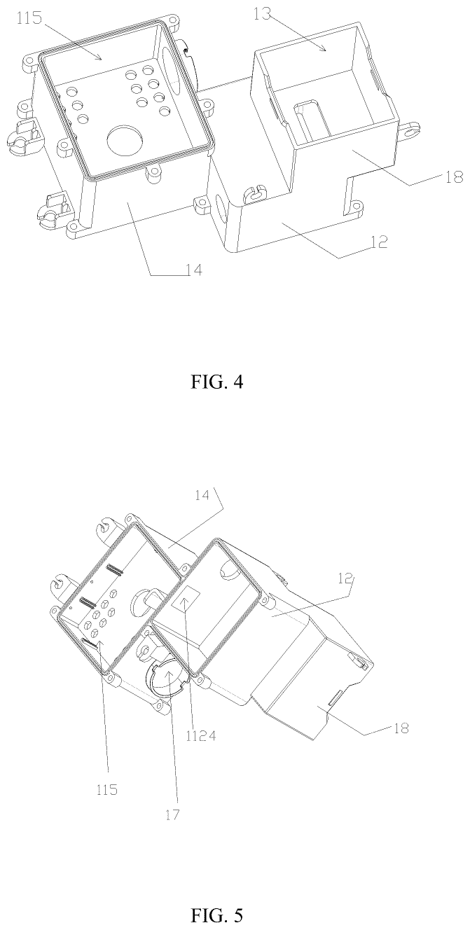

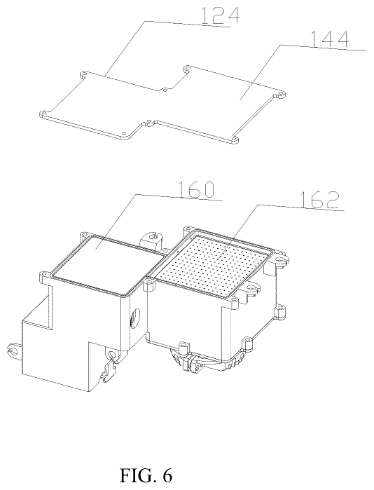

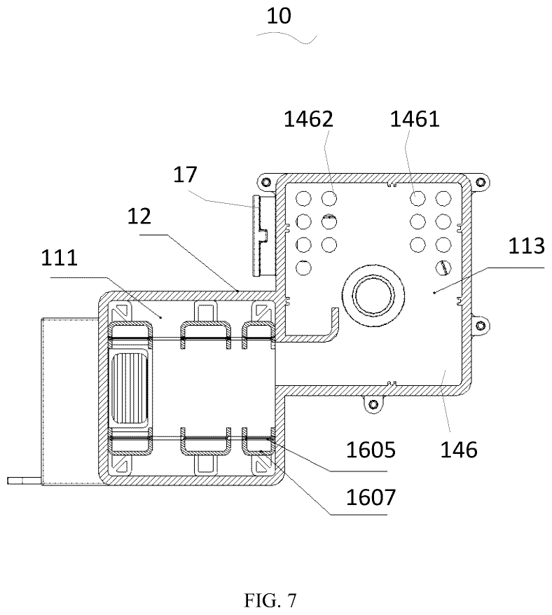

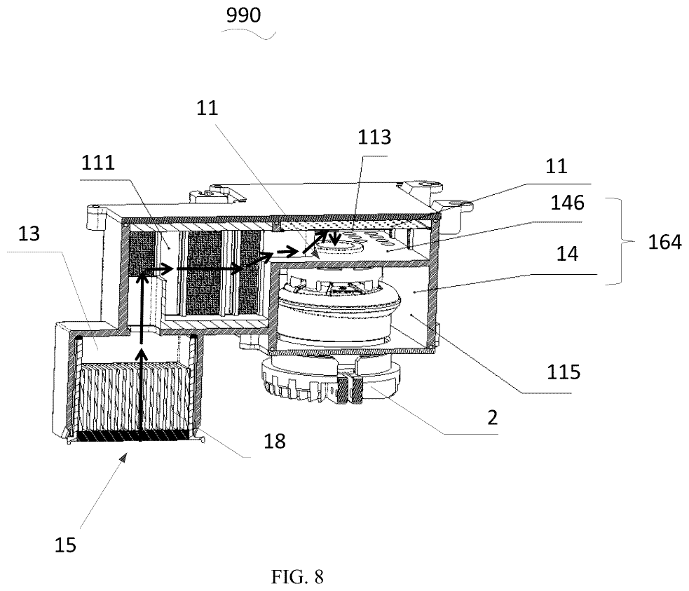

Referring to FIG. 1, a turbine assembly is illustrated. The turbine assembly 990 is contained in a ventilator (not shown). The ventilator includes a ventilator body, and the turbine assembly 990 is installed on the ventilator body. The turbine assembly 990 includes a turbine box 1, and a turbine blower 2 installed in turbine box 1. The turbine blower 2 is configured to process the external airflow entering turbine box 1 to form an airflow with a predetermined flow rate to assist the user in breathing. It can be understood that a predetermined flow rate is an appropriate flow rate for the user's breathing.

The turbine box 1 includes a main body 10, and a silencing chamber 11, a filtering chamber 13, an air inlet 15, and an air outlet 17 defined in the main body 10. The turbine blower 2 is mounted in the silencing chamber 11. The external airflow enters the main body 10 from the air inlet 15 and sequentially passes through the filtering chamber 13 and the silencing chamber 11, and flows out of the main body 10 from the air outlet 17. It can be understood that the air inlet 15 is directly communicated with the filter chamber 13, the air outlet 17 is directly communicated with the silencing chamber 11, and the silencing chamber 11 is directly communicated with the filter chamber 13. The turbine box 1 further includes a silencing structure 16 accommodated in the silencing chamber 11 to attenuate noises generated by the external airflow and vibration of turbine blower 2. The turbine box 1 further includes a filter 130 accommodated in the filter chamber 13 to filter the external airflow.

In this embodiment, the silencing chamber 11 includes a first silencing chamber 111, a second silencing chamber 113, and a third silencing chamber 115. The first silencing chamber 111, the second silencing chamber 113, and the third silencing chamber 115 are sequentially arranged along a direction of the external airflow flowing inside the turbine box 1. The first silencing chamber 111 is communicated with the filtering chamber 13. The third silencing chamber 115 is communicated with the air outlet 17. The second silencing chamber 113 and the third silencing chamber 115 are arranged along a first direction and side by side with the first silencing chamber 111 in a second direction. The first direction and the second direction are perpendicular to each other. In this example, the first direction is vertical and the second direction is horizontal. It can be understood that the first direction and second direction will change with the installation direction of turbine assembly 990, and the above directions are only for example and not limited. It can be understood that the first silencing chamber 111, the second silencing chamber 113, and the third silencing chamber 115 are determined by a structure of the main body 10. The following will provide a detailed description of the structure of the main body 10.

The main body 10 includes a first chamber wall 12, a second chamber wall 14, and a filter chamber wall 18. The first chamber wall 12 surrounds the first silencing chamber 111. The second chamber wall 14 forms a cavity 19. A resonance plate 146 is located in the second chamber wall 14 to separate the cavity 19 into a second silencing chamber 113, and a third silencing chamber 115. The filter chamber wall 16 surrounds the filter chamber 13. The first chamber wall 12 and the filter chamber wall 16 are arranged and connected along the first direction. The first chamber wall 12 and the second chamber wall 14 are arranged and connected along the second direction. A bottom surfaces of the first chamber wall 12 and the second chamber wall 14 are on the same plane, and a top surface of the second chamber wall 14 protrudes relative to the top surface of the first chamber wall 12. The filter chamber wall 16 is located on the top surface of the first chamber wall 12. That is to say, the filter chamber wall 16 is connected to the top surface of the first chamber wall 12, and the second chamber wall 14 is connected to the side surface of the first chamber wall 12.

The filter chamber wall 16 is substantially in a hollow rectangular. The filter chamber wall 18 includes a bottom 181 and a side portion 182 extending vertically from the edge surrounding the bottom 181. The bottom 181 and the side portion 182 form a cavity with an opening, and the opening is facing the bottom 181. The cavity is the filter chamber 13, and the opening is the air inlet 15.

The first chamber wall 12 is substantially in a hollow rectangular. The first chamber wall 12 includes a first top wall 120, a first bottom wall 122 opposite the first top wall 120, and a first sidewall 124 located between the first top wall 120 and the bottom wall 122. The first top wall 120, the first bottom wall 122, and the first sidewall 124 form the first silencing chamber 111. The first top wall 120 is fixedly attached to the bottom 181. The first top wall 120 is misaligned and fits with the bottom 181 only partially fits with the bottom 181. A portion where the first top wall 120 is in contact with the bottom 181 defines a first opening 1126, so that the filter chamber 13 is communicated with the first silencing chamber 111 through the first opening 126. It can be understood that the first sidewall 124 is formed by four rectangular walls 1240 connected in an end-to-end manner.

The second chamber wall 14 includes a second top wall 140, a second bottom wall 142 opposite the second top wall 140, and a second sidewall 144 located between the second top wall 140 and the second bottom wall 142. The second top wall 140, the second sidewall 144 1, and the second bottom wall 122 enclose the cavity 19. The second sidewall 144 is partially attached to the first sidewall 122, and a second opening 1124 is defined in the attach portion between the second sidewall 144 and the first sidewall 122. Therefore, the first silencing chamber 111 and the second silencing chamber 113 can be communicated with each other through the second opening 1124. The second sidewall 144 further defines with an opening, which is the air outlet 17. It can be understood that the second sidewall 144 includes four rectangular walls 1440 connected in connected in an end-to-end manner, and the second opening 1124 and the air outlet 17 are defined in the same wall surface 1440.

The resonance plate 146 is located between the second top wall 140 and the second bottom wall 142, and is connected to the second sidewall 144 along the edge of the resonance plate 146, thereby dividing the cavity 19 into two cavities, one of which is the second silencing cavity 113 and the other is the third silencing cavity 115. The resonance plate 146 is a rectangular flat plate, which is equipped with a first air hole 1461 and a second air hole 1643. In the embodiment, the first pore 1461 and the second pore 1643 respectively include several pores. Therefore, the third silencing chamber 115 and the second silencing chamber 113 are connected through the first air hole 1461 and the second air hole 1643. The first air hole 1461 and the second air hole 1643 are symmetrically located at the installation position of turbine blower 2, respectively. In this embodiment, the second top wall 140 and the first top wall 120 are integrated. In some other embodiments, the second top wall 140 and the first top wall 120 can also be set separately.

The turbine blower 2 is installed on the second top wall 140. When the turbine assembly 990 is in operation, the turbine blower 2 vibrates and external airflow enters in to the turbine box 1. When the external airflow enters the third silencing chamber 115 from the first air hole 1461 and the second air hole 1643, the airflow noise is generated. The turbine blower 2 vibrates and produces vibration noise, and the airflow noise and the vibration noise cause resonance. As a result, the resonance plate 146 and the second chamber wall 14 form a resonance silencing structure 164, to reduce the airflow noise and the vibration noise. It can be understood that the second chamber wall 14 is a resonant wall.

In this embodiment, the silencing structure 16 includes a first noise reduction assembly 160, a second noise reduction assembly 162, and a resonant silencing structure 164. The resonance silencing structure 164 is formed by the resonance plate 146 together with the second chamber wall 14. The structure of the resonance silencing structure 124 is described above and will not be repeated here.

The first noise reduction assembly 160 includes a first mounting plate 1601 and a second mounting plate 1603, a plurality of noise reduction columns 1605 arranged between the first mounting plate 1601 and the second mounting plate 1603 at intervals, and a plurality of silencing cloth 1607 arranged on the noise reduction columns 1605. Each noise reduction column 1605 and corresponding silencing cloth 1607 form a resistive noise reduction structure. The noise reduction columns 1605 are arranged at intervals inside the first silencing chamber 111, forming an expansion type muffling structure with the first silencing chamber 111. The noise reduction columns 1605 are made of flexible silicone, and the silencing cloth 1607 is made of fiberglass mesh material.

The second noise reduction assembly 162 includes a sound-absorbing plate 1622 with several perforations 1620. The sound-absorbing plate 1622 is parallel to the resonance plate 146 and forms a cavity between the second chamber wall 14 to form a resistive noise reduction structure. Specifically, a cavity is formed between the side of the resonance plate 146 away from the turbine blower 2 and the second chamber wall 14, in other words, a resistive noise reduction structure is formed between the side of the resonance plate 146 away from the turbine blower 2 and the second bottom wall 142. The sound-absorbing plate 1622 adopts a PC board.

The turbine blower 2 includes a turbine motor 21, an upper buffer silicone pad 23, and a lower buffer silicone pad 25. The upper buffer silicone pad 23 and the lower buffer silicone pad 25 are respectively positioned on two opposite ends of the turbine motor 21, and the turbine motor 21 is fixed on both sides of the second bottom wall 142 through the upper buffer silicone pad 23 and the lower buffer silicone pad 25.

The above-mentioned turbine assembly have a triple composite sound reduction structure design of resistance expansion chamber resonance chamber, forming a segmented sound reduction effect for high, medium and low frequencies and a step-by-step sound reduction effect. Among them, the first silencing chamber can efficiently control high and medium frequency noise through an impedance composite silencing structure; the fusion of the third and second silencing chambers forms a double-layer vibration chamber for controlling medium and low-frequency noise; the airway diameter inside the turbine box is large, the overall air resistance is small, and it can ensure the power supply of the turbine; the turbo motor forms a double-layer shock absorption and noise reduction through a silicone flexible connection and resonance chamber, providing a double-layer guarantee for stable operation of the turbo motor; the internal structure of the turbine box is set up and a small amount of materials such as silicone and silencing cloth are used for sound-absorbing, forming a good airtightness and sound-absorbing airway assembly. At the same time, the material directly eliminates the use of sound-absorbing cotton materials, thereby reducing the dust particles brought by the cotton and providing a clean and stable airflow.

Certainly, it can be understood that this application is capable of filtering out mid to low frequency noise through a resonant silencing structure. In some other embodiments, the application may also employ soundproofing cotton for sound insulation, disregarding the potential presence of dust particles that soundproofing cotton may introduce. In other words, for the purpose of eliminating the mid to high frequency noise component, existing solutions designed for high frequency noise reduction can be adopted. In essence, the first and second silencing chambers may be omitted, while retaining the resonant silencing structure, specifically the resonant plate and resonant chamber wall.

Obviously, technical personnel in this field can make various modifications and variations to the present application without departing from the spirit and scope of the present application. In this way, if these modifications and variations of the present application fall within the scope of the claims and their equivalent technologies, the present application also intends to include these modifications and variations.

The above listed examples are only the preferred embodiments of this application, and of course, they cannot be used to limit the scope of the rights of this application. Therefore, the equivalent changes made according to the claims of this application still fall within the scope of this application.

1. A turbine box, comprising
a main body, defining a first silencing chamber, a second silencing chamber, a third silencing chamber, an air inlet, and an air outlet; the first silencing chamber being communicated with the air inlet; the third silencing being communicated with the air outlet, each of the first silencing chamber and the second silencing being configured to accommodate a noise reduction assembly, the third silencing chamber being configured to accommodate a turbine blower, external airflow entering to the main body, and passing sequentially through the first silencing chamber, the second silencing chamber, and the third silencing chamber before entering the turbine blower; under an action of the turbine blower, the external airflow is transformed into an airflow with a target flow rate;
a resonance plate, defining a first air hole and a second air hole allowing the external airflow to flow from the second silencing chamber into the third silencing chamber; the first air hole and second air hole being located on opposite sides of the turbine blower; and
a resonance chamber wall, surrounding the third silencing chamber and together with the resonance plate to define the third silencing chamber, an airflow noise generated by the external airflow entering the third silencing chamber through the first air hole and the second air hole, as well as an vibration noise of the turbine blower, causing the resonance plate and the resonance chamber wall to form a resonance silencing structure to silence the airflow noise and the vibration noise.

2. The turbine box of claim 1, wherein the second silencing chamber and third silencing chamber are arranged along a first direction, and arranged side by side with the first silencing chamber in a second direction, the first direction and the second direction are perpendicular to each other.

3. The turbine box of claim 2, wherein the main body comprises a first chamber wall and a second chamber wall connected side by side in a misalignment manner, the first chamber wall encloses the first silencing chamber, the second chamber wall encloses the resonant chamber, the resonant plate is located in the resonant chamber to separate the resonant chamber into the second silencing chamber and the third silencing chamber, and the second chamber wall is the resonant chamber wall.

4. The turbine box of claim 3, wherein the first chamber wall comprises a first sidewall connected to the second chamber wall, the second chamber wall comprises a second sidewall connected to the first chamber wall, the second sidewall defines an air outlet, a portion where the first sidewall and the second sidewall connect defines an opening to enable the second silencing chamber to communicate with the first silencing chamber.

5. The turbine box of claim 4, wherein the noise reduction assembly comprises a first noise reduction assembly and a second noise reduction assembly, the first noise reduction assembly comprises a first mounting plate and a second mounting plate facing to each other, a plurality of noise reduction columns located between the first mounting plate and the second mounting plate, and a plurality of silencing cloth disposed on the noise reduction columns correspondingly, each noise reduction column with the silencing cloth form a resistive noise reduction structure, the noise reduction column and the first silencing chamber form an expansion type muffling structure; the second noise reduction assembly includes a sound-absorbing plate, with a plurality of through hole, the sound-absorbing plate is parallel to the resonant plate, the sound-absorbing plate with the first chamber wall define a cavity to form another resistive noise reduction structure.

6. The turbine box of claim 2, wherein the main body is further defines a filtering chamber, a filter is positioned in the, filtering chamber, the external air is filtered by the filter before enters the first silencing chamber.

7. The turbine box of claim 6, wherein the filter chamber and the first silencing chamber are arranged along the second direction.

8. A ventilator, wherein the ventilator comprises:
a ventilator body,
a turbine, and
a turbine box mounted to the ventilator body, the turbine box comprises:
a main body, defining a first silencing chamber, a second silencing chamber, a third silencing chamber, an air inlet, and an air outlet; the first silencing chamber being communicated with the air inlet; the third silencing being communicated with the air outlet, each of the first silencing chamber and the second silencing being configured to accommodate a noise reduction assembly, the third silencing chamber being configured to accommodate a turbine blower, external airflow entering to the main body, and passing sequentially through the first silencing chamber, the second silencing chamber, and the third silencing chamber before entering the turbine blower; under an action of the turbine blower, the external airflow is transformed into an airflow with a target flow rate;
a resonance plate, defining a first air hole and a second air hole allowing the external airflow to flow from the second silencing chamber into the third silencing chamber; the first air hole and second air hole being located on opposite sides of the turbine blower; and
a resonance chamber wall, surrounding the third silencing chamber and together with the resonance plate to define the third silencing chamber, an airflow noise generated by the external airflow entering the third silencing chamber through the first air hole and the second air hole, as well as an vibration noise of the turbine blower, causing the resonance plate and the resonance chamber wall to form a resonance silencing structure to dampens both the airflow noise and the vibration noise.

9. The ventilator of claim 8, wherein the second silencing chamber and third silencing chamber are arranged along a first direction, and arranged side by side with the first silencing chamber in a second direction, the first direction and the second direction are perpendicular to each other.

10. The ventilator of claim 9, wherein the main body comprises a first chamber wall and a second chamber wall connected side by side in a misalignment manner, the first chamber wall encloses the first silencing chamber, the second chamber wall encloses the resonant chamber, the resonant plate is located in the resonant chamber to separate the resonant chamber into the second silencing chamber and the third silencing chamber, and the second chamber wall is the resonant chamber wall.

11. The ventilator of claim 10, wherein the first chamber wall comprises a first sidewall connected to the second chamber wall, the second chamber wall comprises a second sidewall connected to the first chamber wall, the second sidewall defines an air outlet, a portion where the first sidewall and the second sidewall connect defines an opening to enable the second silencing chamber to communicate with the first silencing chamber.

12. The ventilator of claim 11, wherein the noise reduction assembly comprises a first noise reduction assembly and a second noise reduction assembly, the first noise reduction assembly comprises a first mounting plate and a second mounting plate facing to each other, a plurality of noise reduction columns located between the first mounting plate and the second mounting plate, and a plurality of silencing cloth disposed on the noise reduction columns correspondingly, each noise reduction column with the silencing cloth form a resistive noise reduction structure, the noise reduction column and the first silencing chamber form an expansion type muffling structure; the second noise reduction assembly includes a sound-absorbing plate, with a plurality of through hole, the sound-absorbing plate is parallel to the resonant plate, the sound-absorbing plate with the first chamber wall define a cavity to form another resistive noise reduction structure.

13. The ventilator of claim 9, wherein the main body is further defines a filtering chamber, a filter is positioned in the, filtering chamber, the external air is filtered by the filter before enters the first silencing chamber.

14. The ventilator of claim 13, wherein the filter chamber and the first silencing chamber are arranged along the second direction.

15. A turbine box, comprising a main body containing a resonant silencing structure, the resonant silencing structure comprises:
a resonance plate;
a resonance chamber wall; and
a silencing chamber formed by the resonance plate and the resonance chamber wall, the silencing chamber being configured to install a turbine blower, the silencing chamber being open to outside to receive external airflow, and under an action of the turbine blower, the external airflow is transformed into an airflow with a target flow rate;
wherein the resonance plate defines a first hole and a second air hole; the first and second air holes are located on opposite sides of the turbine blower; an airflow noise generated by the external airflow entering the silencing chamber through the first air hole and the second air hole, along with an vibration noise generated by the turbine blower, induce a resonance effect between the resonance plate and the resonance chamber wall to dampens both the airflow noise and the vibration noise.

16. The turbine box of claim 15, wherein the silencing chamber includes a first silencing chamber, a second silencing chamber, a third silencing chamber; the first silencing chamber being communicated with an air inlet defined in the main body; the third silencing being communicated with an air outlet defined in the main body; each of the first silencing chamber and the second silencing being configured to accommodate a noise reduction assembly; the third silencing chamber being configured to accommodate the turbine blower, external airflow entering to the main body, and passing sequentially through the first silencing chamber, the second silencing chamber, and the third silencing chamber before entering the turbine blower; under an action of the turbine blower, the external airflow is transformed into an airflow with a target flow rate.

17. The turbine box of claim 16, wherein the main body comprises a first chamber wall and a second chamber wall connected side by side in a misalignment manner, the first chamber wall encloses the first silencing chamber, the second chamber wall encloses the resonant chamber, the resonant plate is located in the resonant chamber to separate the resonant chamber into the second silencing chamber and the third silencing chamber, and the second chamber wall is the resonant chamber wall.

18. The turbine box of claim 17, wherein the first chamber wall comprises a first sidewall connected to the second chamber wall, the second chamber wall comprises a second sidewall connected to the first chamber wall, the second sidewall defines an air outlet, a portion where the first sidewall and the second sidewall connect defines an opening to enable the second silencing chamber to communicate with the first silencing chamber.

19. The turbine box of claim 18, wherein the noise reduction assembly comprises a first noise reduction assembly and a second noise reduction assembly, the first noise reduction assembly comprises a first mounting plate and a second mounting plate facing to each other, a plurality of noise reduction columns located between the first mounting plate and the second mounting plate, and a plurality of silencing cloth disposed on the noise reduction columns correspondingly, each noise reduction column with the silencing cloth form a resistive noise reduction structure, the noise reduction column and the first silencing chamber form an expansion type muffling structure; the second noise reduction assembly includes a sound-absorbing plate, with a plurality of through hole, the sound-absorbing plate is parallel to the resonant plate, the sound-absorbing plate with the first chamber wall define a cavity to form another resistive noise reduction structure.

20. The turbine box of claim 17, wherein the main body is further defines a filtering chamber, a filter is positioned in the, filtering chamber, the external air is filtered by the filter before enters the first silencing chamber.
